(19) United States
(12) Patent Application Publication
(10) Pub. No.: US 2006/0141626 A1
(43) Pub. Date: Jun. 29, 2006

(54) Non-random method of gene shuffling

(75) Inventors: Brian M. Hauge, Wildwood, MO (US); Fenggao Dong, Chesterfield, MO (US)

Correspondence Address: HOWREY LLP, C/O IP DOCKETING DEPARTMENT, 2941 FAIRVIEW PARK DRIVE SUITE 200, FALLS CHURCH, VA, 22042, US

(73) Assignee: Monsanto Technology LLC, St. Louis, MO (US)

(21) Appl. No.: 11/258,833

(22) Filed: Oct. 26, 2005

Related U.S. Application Data
(60) Provisional application No. 60/622,450, filed on Oct. 27, 2004.

(51) Int. Cl. C12N 15/87

(52) U.S. Cl. 435/455

(57) ABSTRACT

The present invention concerns the non-random assembling of DNA molecules in a DNA construct and methods of using such constructs, including the production of nucleic acid libraries. The non-random gene shuffling is preferably accomplished by the following steps. First, optionally, the amino acid sequences of proteins encoded by related gene families of interest are aligned and inspected for regions of conserved amino acid residues. These conserved regions, preferably of at least 4 (e.g. about 4 to 10) consecutive conserved amino acid residues are candidate regions for the subsequent design of PCR primers to amplify the variable or less conserved regions in between them, followed by non-random reassembly to create a recombinant nucleic acid genetic library of gene family variants.

This application claims priority to previously filed U.S. provisional application Ser. No. 60/622,450 filed on Oct. 27, 2004, the entire contents of which are incorporated by reference herein.

### FIELD OF THE INVENTION

The present invention relates generally to the field of molecular biology. More specifically, the present invention concerns the assembling of DNA molecules in a non-random order in a DNA construct and methods of using such constructs, including the production of nucleic acid libraries.

### DESCRIPTION OF RELATED ART

Assembly of DNA molecules to create recombinant DNA molecules is well known in the field of molecular biology. Many methods for the creation of recombinant DNA molecules have been developed. For instance, DNA cloning via restriction endonuclease (RE) digestion, followed by ligation of compatible or blunt ends is a well-known method. Other methods include T-A cloning directly from polymerase chain reaction (PCR) products, and ligase-independent cloning (LIC) (Aslanidis and de Jong, NAR 18:6069-6074, 1990), among others. LIC is a highly efficient method to clone complex mixtures of recombinant DNA molecules generated during PCR.

Methods of gene shuffling are also known in the art. These methods rely generally on (a) natural variation or mutagenesis; followed by (b) random recombination or shuffling of DNA fragments to create recombinant DNA molecules and genetic libraries containing those molecules; and (c) selection or screening of these recombinant DNA molecules to identify those with desired properties. For example, U.S. Pat. No. 5,605,793 describes a method of generating randomly recombined DNA molecules. U.S. Pat. Nos. 6,277,632 and 6,495,318 describe a method for linking nucleic acid constructs in a predetermined order.

### SUMMARY OF THE INVENTION

The present invention provides methods for non-random gene shuffling, optionally mediated by ligase independent cloning (LIC), which may be used for the purpose of construction of genetic libraries. The non-random gene shuffling is accomplished by several steps, as outlined in FIG. 1. First, optionally, the amino acid sequences of proteins encoded by related gene families of interest are aligned and inspected for regions of conserved amino acid residues (e.g. by sequence analysis software programs such as the Pretty program of the GCG software package). These conserved regions, preferably of at least 4 (e.g. about 4 to 10) consecutive conserved amino acid residues are candidate regions for the subsequent design of PCR primers to amplify the variable or less conserved regions in between them, followed by non-random reassembly to create a recombinant nucleic acid genetic library of gene family variants.

DNA sequences of the related gene family members possessing regions of variation and conservation in their DNA sequence can be chosen based on the amino acid sequence analysis described above, or based on knowledge of the DNA sequences of the related gene family members. The DNA sequences being shuffled can be discrete domains of multi-domain proteins, or protein fragments. The sequences are then inspected to reveal regions that are convenient for the design of DNA primers. These primers are designed to correspond to conserved regions among the DNA sequences of interest. If desired, mutagenesis can also be conducted to render the analyzed DNA sequences more convenient for primer design. Based on regions of identity of about 7-30 base pairs (bp) or more, sequences are identified for PCR primers that can provide single stranded complementary tails for subsequent cloning via LIC. Alternatively, if ligation or other means are used to generate recombinant DNA molecules, the single stranded complementary regions can be as short as 1 bp long.

The PCR primers are designed in a gene specific manner to the (conserved) sequences abutting the single stranded tails, and PCR is performed using these gene specific primers that contain known tail sequences, 5′ and/or 3′ to the conserved sequences. The sequences of these tail regions in the PCR primers can be identical, or can vary. However, when the tail regions are made single stranded for cloning, each PCR product should preferably have tail regions that are complementary to at least one other tail region on another different PCR product. Additionally, the tail regions should preferably comprise sequences such that annealing to form more than one recombinant annealed product is possible. The PCR reactions can be performed individually for each related gene family member and then the PCR reaction mixture can be subsequently combined with one or more other related gene family member(s) PCR reaction mixtures. Alternatively, the PCR reactions can be performed together, resulting in a complex mixture of PCR products.

The tail regions of the PCR reaction products are then made single stranded by known methods to allow for later hybridization or annealing of complementary strands. For LIC, equimolar amounts of the products are pooled and subjected to LIC. Equimolar amounts are used in an effort to get a random/unbiased assembly. In other words if there are 8 different variants of a fragment in position A, in a population all 8 would be equally represented, assuming there is no other bias. On the other hand, one could bias the population by using different amounts of a product. If conventional ligation is used to join the PCR product fragments, standard protocols may be used. LIC requires at least 7 (preferably up to about 20) overhanging nucleotides to effect joining. One skilled in the art would use ligase for shorter overhangs. If a common region is only 2 nucleotides joining would not be accomplished using LIC, so in vitro ligation would be required. Transformation of the resulting recombinant DNA molecules into E. coli creates a genetic library of non-randomly shuffled variants that can be analyzed by DNA sequencing or used directly for screening or selection, as shown in FIGS. 1 and 2.

This resulting genetic library is considered “shuffled” because PCR products containing complementary single stranded tails can anneal together in multiple arrangements to create novel recombinant DNA molecules. The shuffling is non-random because the location of the DNA sequences where the annealing occurs is controlled by the primer design and the subsequent generation of PCR product molecules being input to the LIC or ligase-dependent cloning procedure. The shuffling pattern may also be controlled by use of tail regions that vary in their ability to anneal together (e.g. are partially or completely non-complementary). Since the primers are designed at discrete positions in the gene(s) of interest the primers specify which segments/regions/domains are shuffled. These regions can be associated with different tails that dictate the order in which the pieces are assembled. For example a given fragment or family of fragments, could be in position 1, or position 2, or position 3. The fragment or family of fragments could also be multeramized etc.

One aspect of this invention provides:

A method for assembling DNA molecules in a non-random order in a DNA construct by

(a) providing at least two double stranded template DNA molecules encoding members of a gene family and possessing regions of variation and of conservation along their DNA sequence;

(b) designing oligonucleotide primers based on conserved sequences between each of the template molecules, wherein the primers also allow for the generation of single stranded 3′ or 5′ nucleic acid tails on an amplified nucleic acid product produced using these primers;

(c) amplifying complementary nucleic acid products of each template DNA molecule using the designed oligonucleotide primers and allowing the complementary nucleic acid products to anneal together to form substantially double stranded nucleic acid molecules;

(d) identifying or creating single stranded 3′ or 5′ single stranded terminal tails on the double stranded nucleic acid molecules, wherein the terminal single stranded nucleic acid tails have a length of from 2 to 30 nucleotides, wherein terminal single-stranded nucleic acid tails on a single double-stranded nucleic acid molecule do not hybridize to each other, wherein a terminal single-stranded nucleic acid tail on a double-stranded nucleic acid molecule is capable of hybridizing to a terminal single-stranded nucleic acid tail extending from a different double-stranded nucleic acid molecule or to a single-stranded DNA oligomer of from about 2 to about 30 nucleotides to allow for assembly of the nucleic molecules in a non-random order; and

(e) incubating said nucleic acid molecules under conditions suitable to promote the assembling of the molecules in a non-random order to create a nucleic acid construct;

wherein there are 2 or more possible orders for the assembly of the nucleic acid molecules.

Another aspect of this invention provides:

A method to create a non-randomly shuffled genetic library of DNA constructs comprising:

(a) utilizing the DNA construct obtained by the method above

(c) cloning the assembled DNA construct into a vector;

(d) transforming a bacterial host with the cloned assembled DNA construct

wherein the vector can replicate autonomously in host cells, and also comprises a selectable or screenable marker and appropriate regulatory signals for expression in a prokaryotic or eukaryotic host cell in which the library may be screened.

In one embodiment of the method, the terminal, single-stranded DNA segments are added during PCR. Oligonucleotides are synthesized to contain a sequence of nucleotides, which is complementary to another terminal, single-stranded DNA segment. Within the oligonucleotide sequence, uridine residues may be substituted for thiamine residues in specific positions. Amplification is performed using a thermal stable polymerase capable of reading through uridine residues in the template. After PCR, the resulting product can be treated with Uracil-DNA glycosylase (UDG), which specifically deaminates the uridine residues. The DNA strand containing the uridine residues becomes unstable after UDG treatment in the positions containing uridine. Following heat treatment, the double-stranded DNA molecule becomes single-stranded in the region containing the uridine residues.

In another embodiment of the method, the single stranded terminal sequences can be created by the method of Jarrell et al (U.S. Pat. No. 6,358,712) using a DNA polymerase that is not able to copy a termination residue of a primer template. In yet another embodiment of the method, a terminal single-stranded DNA segment can be introduced using nicking endoculeases. Nicking endonucleases hydrolyze only one strand of the double-stranded DNA molecule. A nicking endonuclease site can be incorporated into the DNA molecule either through conventional cloning methods available to those skilled in the art or through PCR. Oligonucleotides for PCR can be designed to contain the recognition sequence for any of several commercially available nicking endonucleases. After PCR amplification, the PCR product is treated with the appropriate nicking enzyme. After enzyme treatment, the product is incubated at a temperature sufficient to cause loss of the hydrolyzed strand, resulting in a terminal, single-stranded DNA segment.

In another embodiment of the method, terminal single-stranded DNA segments are introduced by ligation of adapter molecules to the DNA molecule. Assembling of the DNA molecules occurs directly through the hybridization of the terminal single-stranded DNA segments, or an oligomer can be used to bridge two terminal, single-stranded DNA segments.

In another embodiment of this invention, novel proteins are created, for instance by incorporating a DNA sequence encoding an exogenous domain, such as a proline-rich domain, into a shuffled native protein encoding sequence. Alternatively, DNA sequences encoding a native protein domain can be deleted from a shuffled protein encoding sequence, or novel proteins are created by mixing DNA sequences encoding heterologous domains that do not exist together in nature. An example of this would be chimeric transcription factors where you take an activation domain from one transcription factor and fuse it to the DNA binding domain of a second. Entirely novel insecticidal proteins are created by fusing heterologous pore forming domains, with heterologous carbohydrate domains with heterologous lipid binding domains. Another aspect of this invention provides for protein engineering and evolution using a ligase independent cloning system.

### BRIEF DESCRIPTION OF THE DRAWINGS

FIG. 1 illustrates an overview of non-random gene shuffling

FIG. 2 illustrates an overview of non-random gene shuffling with amino acid substitutions and variants created with over-lapping tails.

FIG. 3 illustrates a method of generating hybrid libraries of TIC901 homologs

FIG. 4 shows amino acid sequence alignments of TIC901, TIC1201, TIC407, and TIC417 proteins, and identifies regions of conserved amino acid residues

FIG. 5A-E shows DNA alignments of coding regions for insecticidal proteins

FIG. 6 illustrates a method to increase library diversity by selecting alternative regions for gene shuffling

FIG. 7 illustrates a method for sequential annealing/ligation during library construction

### DETAILED DESCRIPTION OF THE INVENTION

As used herein, “non-random assembly” means that the DNA molecules being joined together via their single stranded termini may become joined together in at least two possible arrangements, orders, or permutations that are governed by the known sequence properties of the termini of these DNA molecules. The order of assembly is not uniquely predetermined, thus allowing for the creation of multiple novel recombinant sequences.

As used herein, the term “assembling” means a process in which DNA molecules are joined through hybridization of terminal, single-stranded DNA segments. The terminal single-stranded DNA segments are preferably non-palindromic sequences, which can be produced by any of several techniques, for instance by PCR, ligation, or chemical treatment of the DNA segments. The terminal single-stranded DNA segments enable users to assemble the DNA molecules in a construct, such as a plasmid.

As used herein, the term “adaptor molecule” means a synthetic oligonucleotide used to attach overhangs to a nucleic acid molecule.

As used herein, the term “DNA construct” refers to a final assembly of the DNA molecules into a plasmid which is capable of autonomous replication within the bacterial hosts, such as Escherichia coli, and may contain elements necessary for stable integration of DNA contained within the vector plasmid into plant host cells.

As used herein, the term “vector” describes a DNA molecule, which contains all of the elements necessary for autonomous replication within bacterial hosts such as Escherichia coli, or Bacillus thuringiensis. The vector also contains a selectable marker for bacterial selection and may contain a different selectable marker used in identifying transformed plant cells.

As used herein, a “region of conservation” of a DNA sequence for the purpose of oligonucleotide primer design is a sequence that encodes at least 4 consecutive identical amino acid residues which is shared among 2 or more DNA sequences being compared to each other.

As used herein, the term “region of variation” of a DNA sequence for the purpose of oligonucleotide primer design refers to a DNA sequence encoding at least 4 amino acids that encodes fewer than 4 consecutive identical amino acid residues when 2 or more DNA sequences are compared to each other.

As used herein, a “gene family” means a group of related genes coding for functionally related proteins or protein domains.

As used herein, a “substantially double stranded” nucleic acid molecule means one that is either entirely double stranded, or is double stranded with the exception of a 1-30 base long 3′ or 5′ single stranded tail region.

As used herein, “exogenous domain” refers to a protein domain found in a protein that is not among the proteins encoded by members of a specific gene family.

As used herein, “native protein” refers to a protein consisting of domains that are normally found together in nature.

As used herein, “heterologous domains” refers to protein domains that do not exist together in nature.

As used herein, “protein” is a polypeptide chain of any size (two or more amino acids lined by a peptide bond.

As used herein, “peptide bond” is the covalent bond between a carbon of one amino acid and the nitrogen of another amino acid where that carbon is referred to in the scientific literature as the Beta carbon and the nitrogen is referred to as the primary nitrogen or N1.

As used herein, “primary structure” means the amino acid sequence of the polypeptide chain in the order they are bound together by peptide bonds.

As used herein, “secondary structure” means the three dimensional shape of a polypeptide chain defined by the angle of carbon and nitrogen backbone of the polypeptide

As used herein, “tertiary structure” means the three dimensional shape of a collection of secondary structures associated together in a single unit or a fold.

As used herein, “domain”, “protein domain”, or “fold” means discrete collections of secondary structures that assume a particular overall shape or tertiary structure.

As used herein, “quaternary structure” means the arrangement and shape of multiple folds either of the same tertiary structure or combinations of multiple tertiary structures.

As used herein, “homologous structural domains” means two or more regions of defined shape and size largely composed of secondary structures that assume an overall similar shape and size. The primary sequence of homologous structural domains are not necessary similar.

As used herein, “protein complex” or “protein pathway” means a collection of proteins that either work together to produce a particular product. This complex or pathway may be composed of multiple homologous and heterologous tertiary and quaternary structures.

As used herein, “organelle” means a collection of diverse proteins and other macromolecules that form together to complete a specific by complex function.

As used herein, “cell” means a collection of organelles and proteins that work together to form a tissue.

As used herein, “tissue” means a collection of cells that associate together to perform a more complex function that a single cell.

As used herein, “organ” means a collection of cells and differentiated tissues associating together to perform a highly complex task.

As used herein, “organism” means an individual cell, collection of cells, collection of tissues, and collection of organs functioning in a coordinated fashion.

As used herein, “population” means a collection of a number of organisms, organs, tissues cells pathways structures, or any collection of anything.

As used herein, the terms “mutation”, “alteration”, “modification” and “substitutions” mean any and all changes to the primary, secondary, tertiary, and quaternary structure of a protein driven by additions, deletions, multiplications, and re-assortments of amino acids, regions of secondary, tertiary and quaternary structure.

As used herein, “protein evolution” means the process of creating and then selecting for mutations with the best outcome for a particular or general function of a protein, protein complex, organelle, cell, tissue, organ, organism, or population.

The present invention has multiple aspects, illustrated by the following non-limiting examples.

### EXAMPLES

### Example 1

### Generation Of Novel Hybrid Insecticidal Toxins

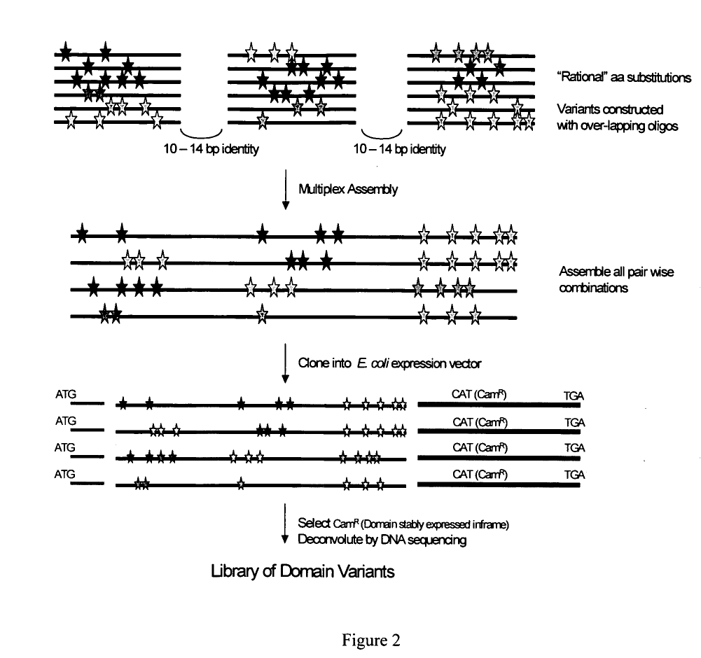

DNA fragments encoding portions of two novel secreted corn rootworm-active Bt toxins (TIC901 and TIC1201) and two novel related secreted proteins (TIC407 and TIC417) can be shuffled in a non-random manner, and used to generate hybrid libraries for subsequent screening in southern and western corn rootworm bioassays in order to select hybrid(s) with improved insecticidal activity. Hybrids are made through generation of PCR fragments between conserved regions of all four proteins followed by re-assembling complete sequences coding for mature hybrid secreted proteins. The hybrids can be expressed in Bt and tested in southern and western corn rootworm bioassays. The overall scheme for generating hybrid libraries is shown on FIG. 2.

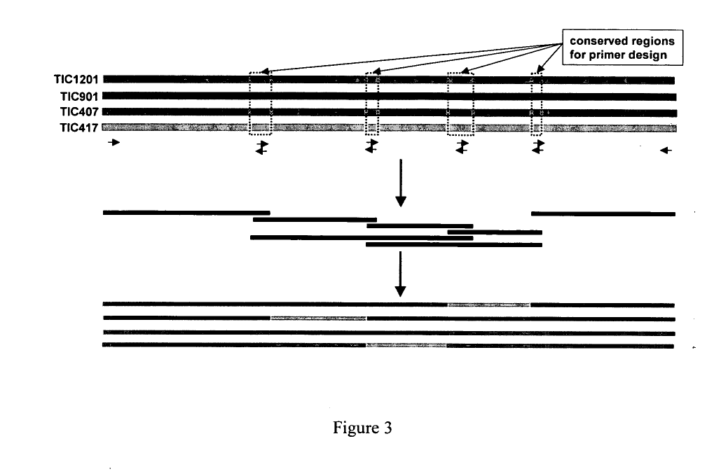

To identify conserved regions to design PCR primers, amino acid sequences of mature TIC901 and TIC1201 proteins, along with predicted mature sequences of TIC407 and TIC417 proteins were subjected to amino acid sequence alignment using Pretty program of the GCG software package. As shown in FIG. 3, examination of the amino acid sequence alignment reveals that there are 10 regions with at least 7 consecutive conserved residues among all 4 sequences. These regions could be used to design PCR primers to amplify the regions in between followed by re-assembly of complete hybrid sequences.

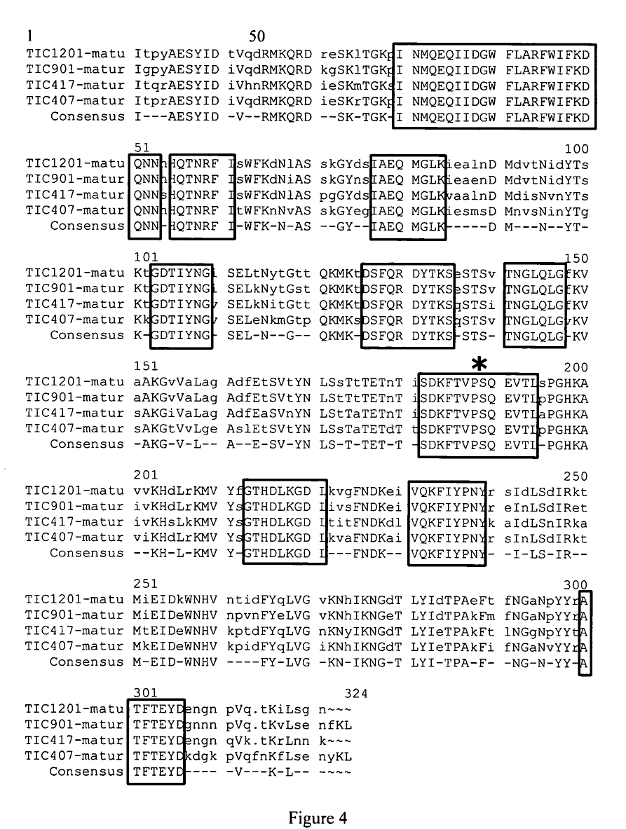

In order to reveal which regions are convenient to design PCR primers, nucleotide alignment of the coding sequences for mature TIC901 and TIC1201 and predicted mature TIC407 and TIC417 was generated using Pretty program of the GCG software package as shown in FIG. 4. The purpose of this alignment was to identify the conserved DNA regions corresponding to conserved protein regions revealed on FIG. 3. Analysis of DNA alignment indicates that, due to degeneracy of the genetic code, among 10 identified conserved protein regions, only three regions are conserved at the DNA level as shown with hatched boxes in FIG. 3, allowing for design of non-degenerate primers.

The fourth highly conserved region on FIG. 3, as shown with solid box labeled with a asterisk in FIG. 3, is rather degenerate at the DNA level. The degeneracy is demonstrated in FIG. 4, underlined and bold). The degeneracy at this region is first removed by PCR mutagenesis, so that all 4 sequences have DNA sequence in this region identical to that of TIC407. A set of complementary pairs of PCR primers to modify DNA sequences of TIC901, TIC1201 and TIC417 in this region are listed below (note that “F’ stands for “forward” primer, “R” stands for “reverse primer”; mutant positions are marked with red color and underlined):

901m-407m-545F. Forward primer for SDKFTVPSQEVT region of TIC901 (SEQ ID NO:1):

5′ - CTG AAA CAA ATA CAA TAT CGG ACA AGT TTA CTG
TCC CAT CCC AAG AAG TTA CAT TGC CTC - 3′

901m-407m-545R. Reverse primer for SDKFTVPSQEVT region of TIC901 (SEQ ID NO:2):

5′ - GAG GCA ATG TAA CTT CTT GGG ATG GGA CAG TAA
ACT TGT CCG ATA TTG TAT TTG TTT CAG - 3′

1201m-407m-545F. Forward primer for SDKFTVPSQEVT region of TIC1201 (SEQ ID NO:3):

5′ - CTG AAA CAA ATA CAA TAT CGG ACA AGT TTA CTG
TCC CAT CCC AAG AAG TTA CAT TAT CCC CAG - 3′

1201m-407m-545R. Reverse primer for SDKFTVPSQEVT region of TIC1201 (SEQ ID NO:4):

5′ - GG ATA ATG TAA CTT CTT GGG ATG GGA CAG TAA
ACT TGT CCG ATA TTG TAT TTG TTT CAG - 3′

417m-407m-545F. Forward primer for SDKFTVPSQEVT region of TIC417 (SEQ ID NO:5):

5′ - CAA CTG AAA CCA ATA CAA TAT CGG ACA AGT TTA
CTG TCC CAT CCC AAG AAG TCA CAT TAG CGC C- 3′

417m-407m-545R. Reverse primer for SDKFTVPSQEVT region of TIC417 (SEQ ID NO:6):

5′ - G GCG CTA ATG TGA CTT CTT GGG ATG GGA CAG TAA
ACT TGT CCG ATA TTG TAT TGG TTT CAG TTG - 3′

After removing degeneracy for the region in red box on FIG. 3, four regions are used to generate PCR fragments covering the regions in between. This can generate a library of 4⁵=1024 possible different clones including 4 original wild-type sequences. The diversity of the library is checked by DNA sequencing, and the whole library is transformed into Bacillus thurigiensis to generate an expression library. Individual clones of that library are screened in southern corn rootworm bioassay to select hybrids with improved southern corn rootworm activity. Hybrids with highest southern corn rootworm activity are tested in western corn rootworm bioassay to select for toxins with improved western corn rootworm activity.

### Example 2

### Construction of a Genetic Library Containing Non-Random Assembled DNA Segments

The assembled DNA constructs of Example 1 may be cloned into a vector and transformed into a host cell, to create a genetic library of non-randomly shuffled gene family variants that may be further analyzed by DNA sequencing, or used directly for screening and selection.

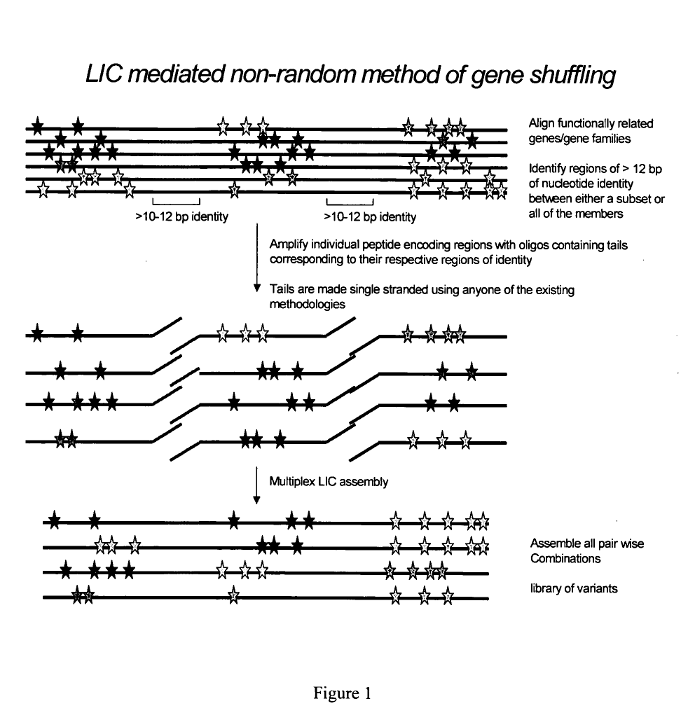

The size and complexity of the library is dictated by the number of individual PCR products from the respective portions of the gene family. If 10 fragments from each of the 3 segments shown in FIG. 1 are used at the start of the procedure, a library with 10³ (1000) variants is produced. If 10 fragments from each of 4 segments are used, 10,000 (10⁴) variants can be produced. By varying the number of input PCR products, direct control over the complexity or diversity of the library is achieved.

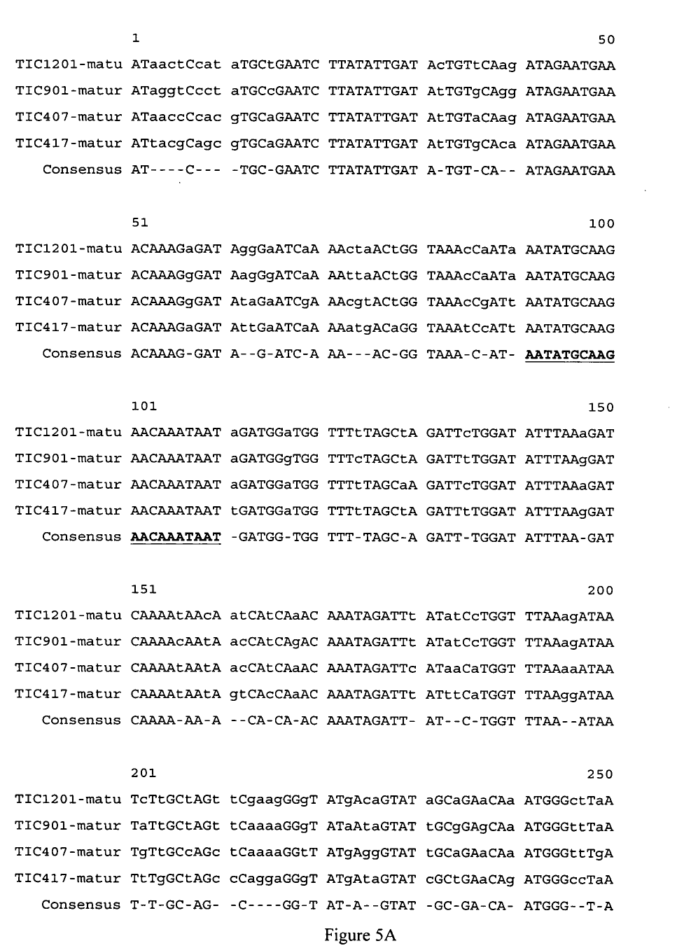

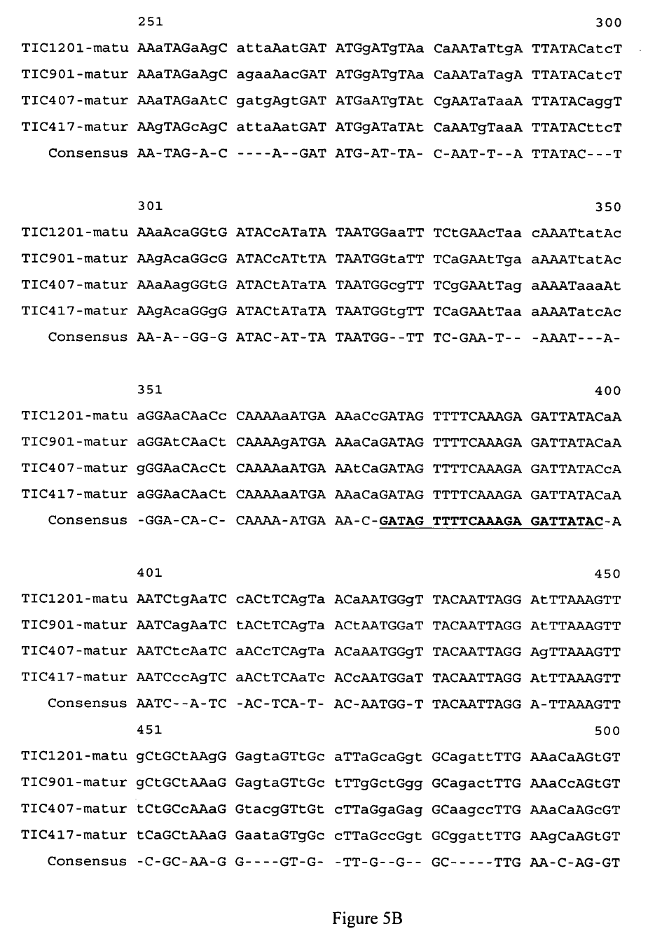

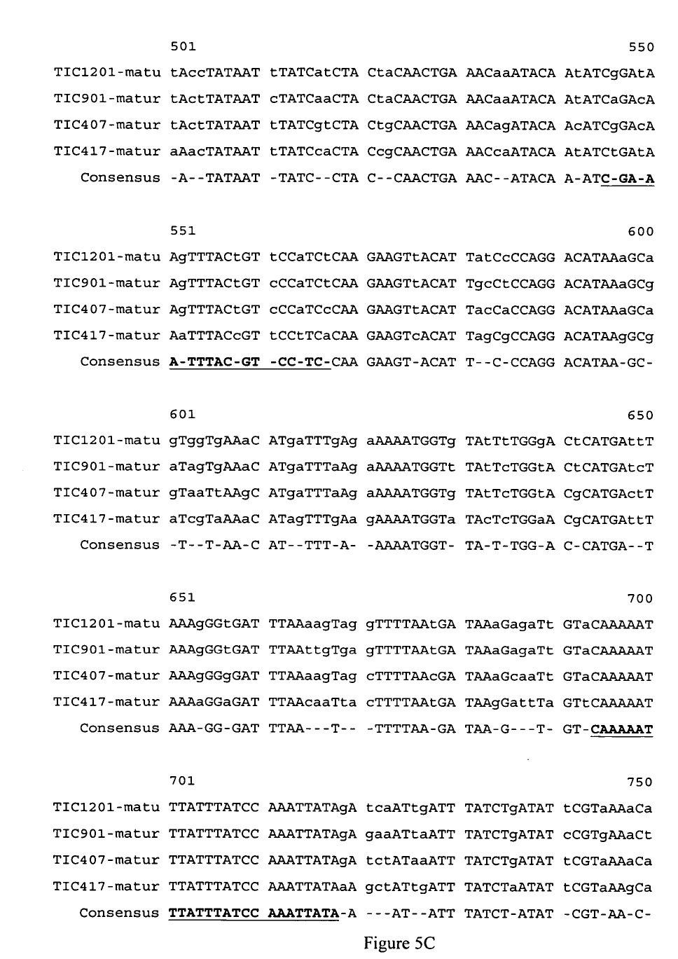

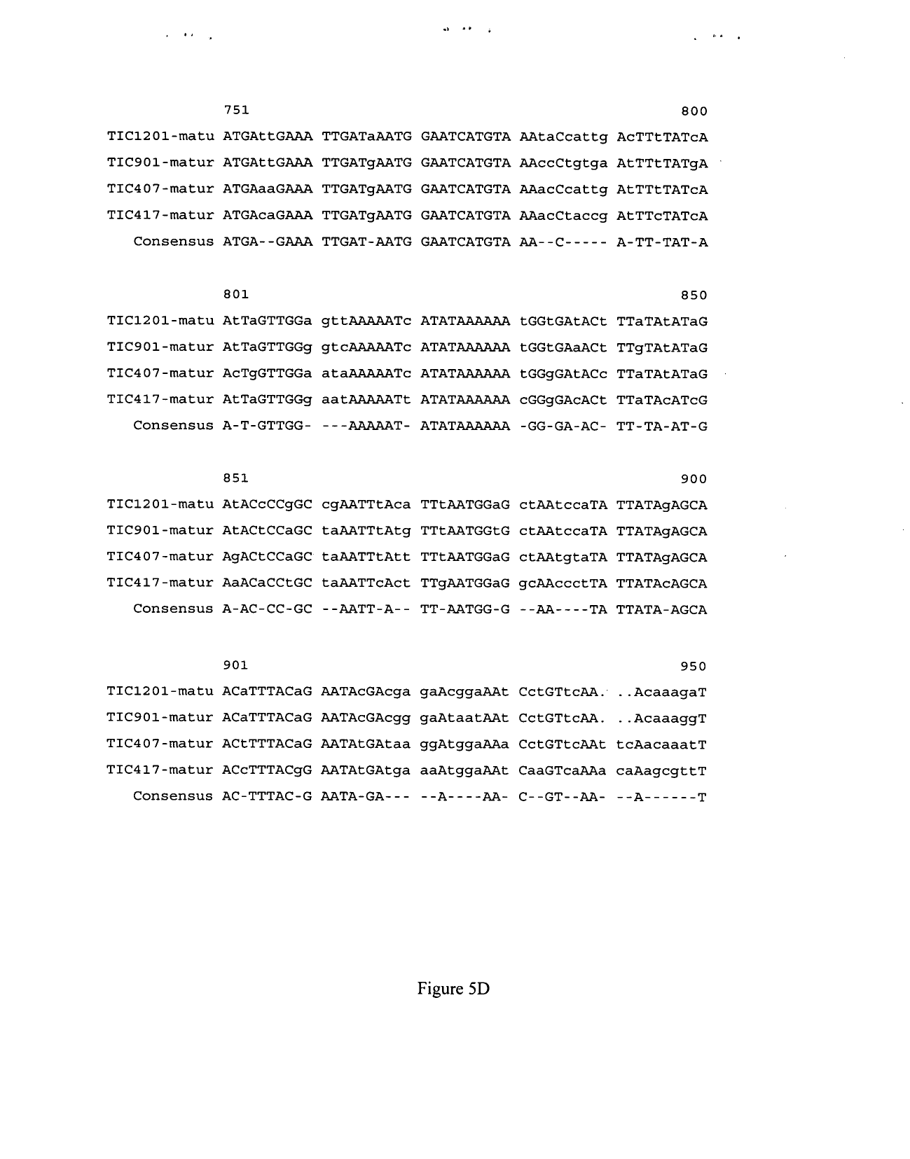

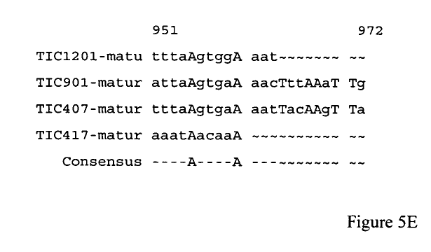

As illustrated in FIG. 5, the diversity can be further increased by selecting alternative regions for non-random shuffling. In practice this may be performed in an iterative fashion. Selected members of library A are shuffled to generate library B, which following selection are used to generate library C. The method is a powerful means to generate large numbers of variants. Because the method is non-random, critical regions of genes encoding an enzyme's active site for instance, are preserved by controlling the input fragments encompassing the critical region.

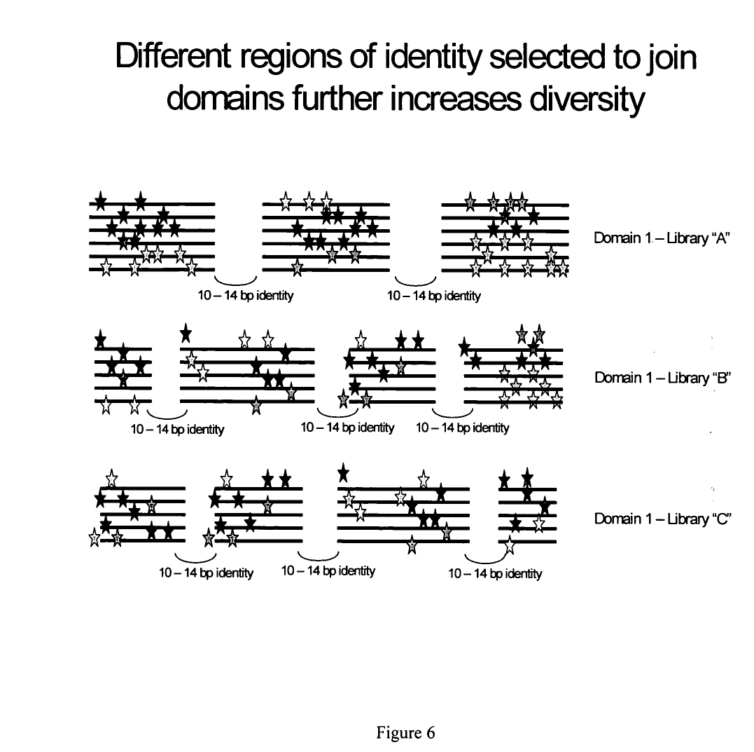

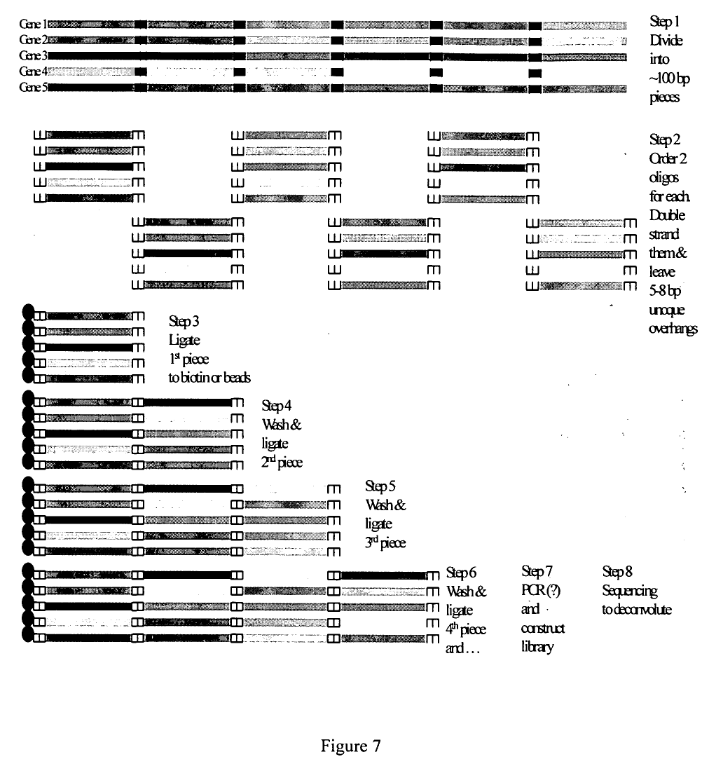

If gene domain shuffling is accomplished via ligation, the assembly of multiple variants may be efficiently carried out in a sequential fashion as shown in FIG. 6. In other words, if there are four pools of DNA molecules (A,B,C,D) to be ligated, A and B would be ligated together, followed by (A+B)+C, and finally (A+B+C)+D. A sequential assembly method could also be employed for LIC mediated assembly by sequentially adding the molecules

### Example 3

### Design of PCR Primers

A set of complementary pairs of PCR primers to generate PCR fragments conserved regions of the four related proteins (TIC1201, TIC901, TIC407, and TIC417) are listed below (note that “F’ stands for “forward” primer, “R” stands for “reverse primer”:

901m-91F Forward primer for QEQIIDGW region (SEQ ID NO:7):

5′ - AAT ATG CAA GAA CAA ATA AT - 3′

901m-91R. Reverse primer for QEQIIDGW region (SEQ ID NO:8):

5′ - AT TAT TTG TTC TTG CAT ATT - 3′

901m-376F. Forward primer for DSFQRDYT region (SEQ ID NO:9):

5′ - GAT AGT TTT CAA AGA GAT TAT AC - 3′

901m-376R. Reverse primer for DSFQRDYT region (SEQ ID NO:10):

5′ - GTA TAA TCT CTT TGA AAA CTA TC - 3′

901m-694F. Forward primer for QKFIYPNY region (SEQ ID NO:11):

5′ - CAA AAA TTT ATT TAT CCA AAT TAT A - 3′

901m-694R. Reverse primer for QKFIYPNY region (SEQ ID NO:12):

5′ - TAT AAT TTG GAT AAA TAA ATT TTT G - 3′

901m-U545F. Forward primer for DKFTVP region (SEQ ID NO:13):

5′ - CGG ACA AGT TTA CTG TCC CAT CC - 3′

901m-U545R. Forward primer for DKFTVPS region (SEQ ID NO:14):

5′ - GG ATG GGA CAG TAA ACT TGT CCG - 3′

### Example 4

### Alternative Method for Hybrid Insecticidal Toxin Library Construction

An alternative way to make TIC901 family hybrid libraries is by choosing only one conserved region of all 4 sequences; for example, the region marked with red asterisk on FIG. 2. This leads to generation of 4²=16 clones (the first wave of hybrids). The clones will be tested in both western and southern corn rootworm bioassays. The results can be analyzed in terms of identifying the regions responsible for improved western and southern corn rootworm activities. Hybrids with highest western and southern corn rootworm activities will be subjected to further hybrid generation across different conserved regions. These steps repeated sequentially leads to the identification of hybrids with improved western and southern corn rootworm activities.

### Example 5

### Protein Engineering and Evolution Using a High Throughput Ligase Independent Cloning System

Protein evolution is the result of evolutionary pressure on metabolic pathways upstream and downstream of the functional role played by a target protein. Thus alterations in one protein can change the evolutionary pressure on a whole set of proteins, such as a regulon. These changes can alter the selection pressure on a whole cell, multiple cells, and, in a multicellular organism, these changes may impact at the tissue and organismal level as well. Additionally, alteration in the behavior of an organism can impact both the population it is a member of, and all levels of the biological hierarchy below it as shown in Table 1.

There are numerous technical methods described in the art for altering the any and all of the structural units or levels of structure. Any and all of these methods can be used with ligase independent cloning to effect the production of genetic alterations that translate into altered protein structure and subsequently impacting the structure of organelles, cells, tissues, organs, organisms and populations. See Table 1. These methods include:

1. Methods for adding or deleting an amino acid or sequence of amino acids to a primary structure.

2. Methods for substituting one amino acid for another in an amino acid primary structure.

3. Methods for prediction the best amino acid addition, deletion, or substitution to the primary structure.

4. Methods for preventing premature termination of the amino acid structure.

5. Methods for adding, deleting, or modifying a region of secondary structure.

6. Methods for predicting the best addition, deletion or substitution of secondary structure.

7. Methods for adding, deleting or modifying a region of tertiary structure.

8. Methods defining and adding liking or intervening sequences between units of tertiary structure so as to permit effective construction of a protein with homologous or heterologous domains.

9. Methods for predicting the best mutation to the quaternary structure

10. Methods for altering the quaternary structure of a protein including the position of one domain relative to another as modified by intervening sequences or linkers.

11. Methods for altering the quaternary structure of a protein

12. Methods for predicting the best alteration to the quaternary structure

13. Methods for altering the genetic make-up of a cell, organelle, or organ.

14. Methods of altering the genetic make-up of an organism

15. Methods for mutating a cell or organism

16. Methods for predicting the best mutations to a cell, organelle, tissue, cell or organism.

17. Methods for altering the genetic make-up of a population

18. Methods for predicting the best genetic make-up of a population.

19. Methods for altering the relationship of one organism with another or one population of organisms with another population of organisms.

20. Methods for altering the relationship of one cell with another cell, either of the same cell type or any other cell.

All of these methods can be used with Ligase Independent Cloning to drive the evolution of proteins and higher order structures composed at least in part of proteins.

TABLE 1
The set of possible mutations, units of mutation and impacts

| Structure | | Impact | | |
|---|---|---|---|---|
| unit or | Units of | on Other | Example of | |
| Level of | Mutation/ | Struc- | Technical | Example |
| structure | alteration | tures | method | of use |
| Primary | Amino acids | All | U.S. Pat. No. | Cry3Bb |
| | | levels of | 006077824A | |
| | | structure | | |
| Secondary | Amino acids, | All | (Layfield et | Paget's |
| | Units of | levels of | al., 2004) | disease |
| | secondary | structure | Agarkov et | Tummo- |
| | structure | | al., 2004) | tifs |
| Tertiary | Amino acids, | All | (U.S. Pat. No. | Cry3Bb |
| | Units of sec- | levels of | 006077824A) | |
| | ondary struc- | structure | (Apic et al., | |
| | ture, Units of | | 2001) | |
| | tertiary | | | |
| | structure | | | |
| Quaternary | Amino acids, | All | (Perham, 2000) | Lipid |
| | Units of sec- | levels of | | metabo- |
| | ondary struc- | structure | | lism. |
| | ture, Units of | | | |
| | Tertiary Struc- | | | |
| | ture Units of | | | |
| | quaternary | | | |
| | structure | | | |
| Pathway/ | All previous | All | (Rui et al., | Degrada- |
| Protein | levels and | levels of | 2004) | tion of |
| complex | other pathways | structure | | chlori- |
| (Pathway | or protein | | | nated |
| or complex | complexes | | | hydro- |
| engineering | | | | carbons |
| Organelle | All previous | All | (Spirek et | mitochon- |
| Organelle | levels and | levels of | al., 2001) | dria |
| engineering | other macro- | structure | | |
| | molecules | | | |
| Cell | All previous | All | (Petri and | Removing |
| (Cell | levels | levels of | Schmidt - | cell |
| Engineering) | And organelles | structure | Dannert 2004) | contact |
| | | | | inhibition, |
| | | | | white |
| | | | | cell pro- |
| | | | | liferation |
| Tissue | All previous | All | (Bartholomew | Cultured |
| (Tissue | levels and | levels of | et al., 2002) | epithelial |
| Engineering) | cells | structure | (Brittberg et | cells |
| | | | al., 2001) | Cartilage |
| | | | | repair |
| Organ (Organ | All previous | All | (Ball and | Organ |
| Engineering) | levels and | levels of | Barber, 2003) | culture |
| | organs | structure | | |
| Organism | All previous | All | (Loi et al., | Organism |
| (Organism | levels and | levels of | 2001) | cloning |
| engineering) | organisms | structure | | |
| Population | All previous | All | (Kuzovkina, | Plant |
| (Population | levels and | levels of | et al., 2004) | root- |
| engineering) | populations | structure | | rhyzo- |
| | | | | sphere |
| | | | | inter- |
| | | | | actions |

### REFERENCES

U.S. Pat. No. 5,605,793. Methods for in vitro recombination, Stemmer W.

U.S. Pat. No. 6,277,632. Method and kits for preparing multicomponent nucleic acid constructs, Harney P. D.

U.S. Pat. No. 6,495,318. Method and kits for preparing multicomponent nucleic acid constructs, Harney P. D.

U.S. Pat. No. 6,077,824. Methods for improving the activity of .delta.-endotoxins against insect pests, English L., et al.

U.S. Pat. No. 6,358,712. Ordered gene assembly, Jarrell K., et al.

U.S. Pat. No. 6,077,824. English, L. H., Brussock, S. M., Malvar, T. M., Bryson, J. W., Kulesza, C. A., Walters, F. S., Slatin, S. L., Von Tersch M. A. 2000. Methods for improving the activity of delta-endotoxins against insect pests.

Agarkov, A., Greenfield, S. J., Ohishi, T. et al. 2004. Catalysis with phosphine-containing amino acids in various “turn” motifs. J. Org. Chem. 69, 8077-8085.

Apic, G., Gough, J., Teichmann, S. A. 2001. Domain combinations in archael, eubacterial and eukaryotic proteomes. J. Mol. Biol. 301, 311-325.

Aslanidis and P J de Jong. 1990. Ligation-independent cloning of PCR products (LIC-PCR). Nucl. Acids Res. 18, 6069-6074.

Ball, S. G., Barber, T. M., 2003. Molecular development of the pancreatic beta cell: implications for cell replacement therapy. Trends in endocrinology and metabolism 14, 349-355.

Bartholomew, A., Sturgeon, C., Siatskas, M., Ferrer, K., McIntosh, K., Patil, S., Hardy, W., Divine, S., Ucker, D., Deans, R., Moseley, A., Hoffman, R. 2002. Mesenchymal stem cells suppress lymphocyte proliveration in vitro and prolong skin graft survival in vivo. Experimental Hematology 30, 42-48.

Brittberg, L., Tallheden, T., Sjogren-Jansson E., Lindahl, A., and Peterson, I. 2001. Autologous chondrochtes used for articular cartilage repair—an update. Clinical Orthopaedics and Related Research, 391, S337-S348.

Layfield, R., Ciani, B., Ralston, S. H., Hocking, L. J., Sheppard, P. W., Searle, M. S., Cavey, J. R. 2004. Structural and functional studies of mutation affecting the UBA domain of SQSTM1 which causes Paget's disease of bone. Biochemical Society Transactions 32, 728-730.

Loi, P., Ptak, G., Barboni, B., Fulka, J., Cappai, P., Clinton, M. 2001. Genetic rescue of an endangered mammal by cross-species nuclear transfer using post-mortem somatic cells. Nature Biotechnology, 19, 962-964.

Perham, N. 2000. Swinging arms and swinging domains in multifunctional enzymes: Catalytic machines or multistep reactions. Annu Rev., Biochem. 69, 961-1004.

Petri, R. and Schmidt-Dannert, C., 2004. Dealing with complexity: evolutionary engineering and genome shuffling. Current Opinion in Biotechnology 15, 298-304.

Kuzovkina, L N., AI'terman, I. E., Karandashov, V. E. 2004. Genetically transformed plant roots as model for studying specific metabolism and symbiotic contacts of the root system. Biological Bulletin 31, 255-261.

Rui, L. Y., Kwon, Y. M., Reardon, K. F. 2004. Metabolic pathway engineering to enhance aerobic degradation of chlorinated ethenes and to reduce their toxicity by cloning a novel glutathione S-transferase, an evolved toluene o-monooxygenase, and gamma glutamylcysteine synthetase. Environ Microbiol 6, 491-500.

Spirek, M., Polakova, S., Skutova, D. Yeast organelle engineering II. How the alien mitochondria and nuclei get together. Yeast 18, S123-S123.

What is claimed is:

1. A method for assembling DNA molecules in a non-random order in a DNA construct by
(a) providing at least two double stranded template DNA molecules encoding members of a gene family and possessing regions of variation and of conservation along their DNA sequence;
(b) designing oligonucleotide primers based on conserved sequences between each of the template molecules, wherein the primers also allow for the generation of single stranded 3′ or 5′ nucleic acid tails on an amplified nucleic acid product produced using these primers;
(c) amplifying complementary nucleic acid products of each template DNA molecule using the designed oligonucleotide primers and allowing the complementary nucleic acid products to anneal together to form substantially double stranded nucleic acid molecules;
(d) identifying or creating single stranded 3′ or 5′ single stranded terminal tails on the double stranded nucleic acid molecules, wherein the terminal single stranded nucleic acid tails have a length of from 2 to 30 nucleotides, wherein terminal single-stranded nucleic acid tails on a single double-stranded nucleic acid molecule do not hybridize to each other, wherein a terminal single-stranded nucleic acid tail on a double-stranded nucleic acid molecule is capable of hybridizing to a terminal single-stranded nucleic acid tail extending from a different double-stranded nucleic acid molecule or to a single-stranded DNA oligomer of from about 2 to about 30 nucleotides to allow for assembly of the nucleic molecules in a non-random order; and
(e) incubating said nucleic acid molecules under conditions suitable to promote the assembling of the molecules in a non-random order to create a nucleic acid construct;
wherein there are 2 or more possible orders for the assembly of the nucleic acid molecules.

2. The method of claim 1, wherein the amplified nucleic acid comprises nucleic acids selected from one or more of the group comprising DNA, RNA, and DNA comprising one or more modified bases.

3. The method of claim 1, wherein the oligonucleotide primer comprises nucleic acids selected from one or more of the group comprising DNA, RNA, and DNA comprising one or more modified bases.

4. The method of claim 1, wherein the double stranded template molecule encodes a multidomain protein

5. The method of claim 1 wherein the double stranded template molecule encodes a single protein domain.

6. The method of claim 1 wherein the 3′ or 5′ terminal group of the amplified nucleic acid is phosphorylated.

7. The method of claim 1 wherein the nucleic acid molecules are annealed in the absence of DNA ligase.

8. The method of claim 1 wherein the nucleic acid molecules are annealed in the presence of DNA ligase.

9. The method of claim 1 wherein the template DNA sequences are derived from Bacillus thuringiensis.

10. The method of claim 8 wherein the assembled nucleic acid construct encodes a protein toxic to a dipteran insect, a lepidopteran insect, a coleopteran insect, or a nematode.

11. A method to create a non-randomly shuffled genetic library of DNA constructs comprising:
(a) utilizing the DNA construct obtained in any of claims 1-10
(c) cloning the assembled DNA construct into a vector;
(d) transforming a bacterial host with the cloned assembled DNA construct
wherein the vector can replicate autonomously in host cells, and also comprises a selectable or screenable marker and appropriate regulatory signals for expression in a prokaryotic or eukaryotic host cell in which the library may be screened.


SEQUENCE LISTING (14 sequences)

SEQ ID NO: 1 - DNA, 60 bp, Bacillus thurigiensis
ctgaaacaaatacaatatcggacaagtttactgtcccatcccaagaagttacattgcctc

SEQ ID NO: 2 - DNA, 60 bp, Bacillus thurigiensis
gaggcaatgtaacttcttgggatgggacagtaaacttgtccgatattgtatttgtttcag

SEQ ID NO: 3 - DNA, 63 bp, Bacillus thurigiensis
ctgaaacaaatacaatatcggacaagtttactgtcccatcccaagaagttacattatccccag

SEQ ID NO: 4 - DNA, 59 bp, Bacillus thurigiensis
ggataatgtaacttcttgggatgggacagtaaacttgtccgatattgtatttgtttcag

SEQ ID NO: 5 - DNA, 64 bp, Bacillus thurigiensis
caactgaaaccaatacaatatcggacaagtttactgtcccatcccaagaagtcacattagcgcc

SEQ ID NO: 6 - DNA, 64 bp, Bacillus thurigiensis
ggcgctaatgtgacttcttgggatgggacagtaaacttgtccgatattgtattggtttcagttg

SEQ ID NO: 7 - DNA, 20 bp, Bacillus thurigiensis
aatatgcaagaacaaataat

SEQ ID NO: 8 - DNA, 20 bp, Bacillus thurigiensis
attatttgttcttgcatatt

SEQ ID NO: 9 - DNA, 23 bp, Bacillus thurigiensis
gatagttttcaaagagattatac

SEQ ID NO: 10 - DNA, 23 bp, Bacillus thurigiensis
gtataatctctttgaaaactatc

SEQ ID NO: 11 - DNA, 25 bp, Bacillus thurigiensis
caaaaatttatttatccaaattata

SEQ ID NO: 12 - DNA, 25 bp, Bacillus thurigiensis
tataatttggataaataaatttttg

SEQ ID NO: 13 - DNA, 23 bp, Bacillus thurigiensis
cggacaagtttactgtcccatcc

SEQ ID NO: 14 - DNA, 23 bp, Bacillus thurigiensis
ggatgggacagtaaacttgtccg